(19) United States
(12) Patent Application Publication
(10) Pub. No.: US 2015/0011980 A1
(43) Pub. Date: Jan. 8, 2015

(54) Body contour-fit wound dressing for vacuum therapy

(71) Applicant: Hong Tan, Xi'an (CN)

(72) Inventors: Hong Tan, XI'AN (CN); Jinqing Li, Xi'an (CN)

(21) Appl. No.: 13/933,129

(22) Filed: Jul. 2, 2013

(51) Int. Cl. A61F 13/00

(52) U.S. Cl. CPC A61F 13/00068
USPC 604/544; 604/319

(57) ABSTRACT

A wound dressing is provided, for the vacuum treatment of wounds by connecting to a vacuum source. The wound dressing comprises an airtight outer layer, a body contour-fit layer with multi pores, a body contour-fit tubing system, a non-adherent wound contacting layer and the pendent membranes with adhesives around the margin of the wound dressing. The said body contour-fit layer is separated by airtight membrane to obsolete cells. All the said parts of the dressing are integrated to one piece. The body contour-fit layer and the tubing system are molded or tailored to fit and cover part of, one or several body regions. Upon application of the said dressing, no assembly or trim of the said dressing is needed.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND AND OF THE INVENTION

Many prior art references presented evidences that the provision of the vacuum in the space above the surface of a chronic wound or an infected wound facilitates wound healing. In the 1980s, Russian doctor Davydov et al first established the efficacy of vacuum therapy, which is also called negative pressure wound therapy (NPWT), on wound healing¹⁻³. It has been proven that the vacuum therapy enhances the wound healing of the chronic wounds, the infected wounds and the war wounds⁴⁻⁹. In addition, the vacuum therapy is becoming the routine for preparation of almost all kinds of wounds prior to grafting or flaps for wound closure.

The rational of the typical vacuum therapy is as follows: the wound is covered by multi-pore material, usually foam or gauze; an outer layer of membrane, which is airtight but maybe water vapor permeable, seals the multi-pore material and the wound; the space which is sealed by the airtight membrane is connected by a tube to a vacuum source, and subsequently negative pressure is formed on the surface of the wound. All the commercially-available dressings for vacuum therapy contain these 3 said separate parts, including the multi-pore material, the outer layer of membrane and the tube. However, there are differences in these parts among different brands. For example, the multi-pore material of the V.A.C.® GranuFoam Dressings (KCI Medical Ltd) is polyurethane (PU) foam, and that of V.A.C.® WhiteFoam Dressings (KCI Medical Ltd) is the polyvinyl alcohol (PVA) foam; while that of V1sta™ dressings (Smith and Nephew), Venturi™ dressings (Talley Medical), Exsudex™ dressings (Synergy), WoundAssist™ dressings (Huntleigh Healthcare), Genadyne™ dressings (Genadyne Europe BV) and Invia Liberty™ dressings (Medela Healthcare) is gauze.

There are several shortcomings of current commercially-available dressings for the vacuum therapy. First of all although the companies have developed many special designs, like the “Spiral-cut foam” design of KCI medial Ltd to simplify the sizing of the foam, it is time-consuming to apply commercially-available vacuum dressings on the big wounds. Since the different parts of the commercially available dressing are separate, the medical healthcare givers need to trim the multi-pore material (either foam or gauze) to fit the wound, adhere the airtight membrane to the healthy skin adjacent to the wound to seal the said multi-pore material, and then connect the tube to the space and the vacuum source.

Secondly, some wounds just cannot be covered by one dressing although the companies have different sizes for their vacuum therapy dressings. For example, in some adult patients with skin defect on the whole leg, 2 or more dressings are needed to cover the wound and it would be time-consuming to patch up these dressings on the wound.

In addition, the current commercially-available foam-based vacuum dressings are plate-like without curves, while all of the human skin or wounds have curves. It will be especially clumsy to apply foam-based vacuum dressings on the position with complicated contour, such as applying the said foam-based dressings to cover the whole foot. There is only one exception that KCI Medical Ltd has developed V.A.C.® GranuFoam™ Hand Dressing to fit the hand contour of the patient. However, this said hand contour-fit dressing contains 3 separate parts, i.e. the foam, the sealing bag and the tube (so called SensaT.R.A.C. Pad™), which means the caregivers have to assembly these part when using this dressing. In clinical setting, 2 kinds of injuries, which are the most happened injuries involved the whole or most of the hand, are the burn injury and the severe avulsion injury of hand. In the management of these 2 said injuries of hand, no published clinical report has shown evident benefit of application of V.A.C.® GranuFoam™ Hand Dressing. Because the vacuum therapy has physical pressure on the wound, the vacuum dressing for hand might worse the impaired circulation of the hands under these 2 said injuries.

Furthermore, application of current commercially-available vacuum dressing is not practical for the wounds in certain areas, such as the decubitus ulcers adjacent to anus. It is imperative for the current. commercially available vacuum dressing having the airtight membrane adhere to the healthy skin to seal the said dressing on the wound. Otherwise, negative pressure cannot be formed in the space between the airtight membrane and the wound. In the patient with the decubitus ulcers, the skin around 5 cm along the wound edge is unhealthy and always has signs including hyperkeratosis, edema, and humidity. The airtight membrane cannot adhere to the said skin. In addition, the airtight membrane cannot be applied on the skin 3 cm around the anus either. That means the decubitus ulcers at the ischial tuberosity cannot be treated with the vacuum therapy, because the distance between the said ischial tuberosity and the anus is around 5 cm.

### REFERENCE

1. Davydov Iu A, Lariehev A B, Smimov A P, Flegontov V B. [Vacuum therapy of acute suppurative diseases of soft tissues and suppurative wounds]. Vestn Khir Im I I Grek. 1988; 141(9):43-46.
2. Davydov Iu A, Larichev A B, Kozlov A G. [Pathogenetic mechanisms of the effect of vacuum therapy on the course of the wound process]. Khirurgiia (Mosk). 1990(6):42-47.
3. Davydov In A, Malafeeva E V, Smimov A P, Flegontov V B. [vacuum therapy in the treatment of suppurative lactation mastitis]. Vestn Khir Im I I Grek. 1986; 137(11):66-70.
4. Egemen O, Ozkaya O, Ozturk M B, Aksan T, Oman C, Akan M. Effective use of negative pressure wound therapy provides quick wound-bed preparation and complete graft take in the management of chronic venous ulcers. Int Wound J. 2012:9(2): 199-205.
5. Damiani G, Pinnarelli L, Sommella L, et al. Vacuum-assisted closure therapy for patients with infected sternal wounds: a meta-analysis of current evidence. J Plast Reconstr Aesthet Surg. 2011:64(9):1119-1123.
6. Dunn R, Hurd T, Chadwick P, et al. Factors associated with positive outcomes in 131 patients treated with gauze-based negative pressure wound therapy. Int J Surg. 2011; 9(3):258-262.
7. Hurd T, Chadwick P, Cote J, Cockwill J, Mole T R, Smith J M. Impact of gauze-based NPWT on the patient and nursing experience in the treatment of challenging wounds. Int Wound J. 2010; 7(6):448-455.
8. Nather A, Chionh S B, Han A Y, Chan P P, Nambiar A. Effectiveness of vacuum-assisted closure (VAC) therapy in the healing of chronic diabetic foot ulcers. Ann Acad Med Singapore. 2010; 39(5):353-358.
9. Powell ETt. The role of negative pressure wound therapy with reticulated open cell foam in the treatment of war wounds. J Orthop Trauma. 2008; 22(10 Suppl):S138-141.
10. Fraccalvieri M, Zingarelli E, Ruka E, et al. Negative pressure wound therapy using gauze and foam: histological, immunohistochemical and ultrasonography morphological analysis of the granulation tissue and scar tissue. Preliminary report of a clinical study. Int Wound J. 2011; 8(4):355-364.

### BRIEF SUMMARY OF THE INVENTION

Please note that the body regions mentioned hereafter in this application, including the neck, the shoulder, the chest, the abdomen, the back, the hips, the perineal region, the thigh, the knee, the leg, the ankle, the foot, the armpit, the arm, the elbow, the forearm and the wrist, are correlated to one or several known human anatomical regions. Please refer to ‘Detailed Description of the Invention’ for details.

The invention in this application is designed to address the shortcomings of said current commercially-available vacuum dressings in the ‘Background of the Invention’. The present invention relates to dressings for wounds, and more particularly to the provision of dressing for use with a vacuum source. The dressing comprises a body contour-fit layer which is made of multi-pore material and is casted or tailored to fit and cover one (including part of) or several body regions of the patients; an outer membrane which is airproof and waterproof (the membrane may be water vapor permeable), a wound contact layer which is thin, non-adherent to the wound, and has multi pores; and a tubing system which locates between the body contour-fit layer and the outer layer and connects to the vacuum source. In addition, the body contour-fit layer is separated by airproof and waterproof membranes into obsolete cells, which connected by the tubing system to the vacuum source. The dressing may have one or two openings via which the dressing is applied on the body region(s). On all the margins of the dressing and one side of the opening(s), there are the pendent membranes with viscous adhesive which is protected by the protective membranes. All the said parts of the present invention are integrated to one piece.

Since the dressing will cover one (including part of) or several anatomical regions, there is normal skin along with the wound(s) covered by the dressing. To avoid or lessen the soakage of skin by the wound fluid, the body contour-fit layer of the present invention is separated by air- and water-proof membrane into isolate cells, which will confine the wound fluid in the cells which are over the wound(s).

The embodiments of the invention will save the caregivers' time to apply the vacuum dressings to big wounds. For instance, the embodiment shown in FIG. 12, which covers the whole leg and the knee, can be used to treat the big wound in the leg. Since the outer layer, the body contour-fit layer and the tubing system of the said dressing are integrated into one piece, caregivers may just put the leg into the said dressing via the longitude opening, peel of the protective membrane on the pendent membrane, adhere the said pendent membrane to the skin and opening, and then connect the tube to the vacuum source. Unlike the current commercially-available vacuum dressings, caregivers don't have to assembly the dressing and don't have to patch up 2 or several dressings to cover the wound. In addition, the embodiments of the invention will be beneficial to treat war wounds in battle field or under terror attack, where the number of patients may outweigh the available medical sources during the relative short time and the delay of the treatment to war injury often means infection.

The embodiments of the invention will save the caregivers' time to apply the vacuum dressings to the human region which has complicated contour. For example, the embodiment shown in FIG. 8 can be used to cover the big wound on the foot and the ankle. Since the said dressing is integrated into one piece, caregivers may just put the foot into the said dressing via the longitude opening, have the pendent membrane adhere to the skin and the opening, and then connect the tube to the vacuum source. While application of the current commercially-available vacuum dressings will be clumsy because they have to be assembled and they have to be bent to fit the contour of the foot.

Some embodiments of this invention can be used to cover the regions which cannot be covered by the current commercially-available dressings. For example, the embodiment which shown in FIG. 16A and FIG. 16B, which cover the hips, the perineal region, part of the abdomen and part of the thighs, can be used to treat the decubitus ulcers at the ischial tuberosity, which cannot be treated by the current commercially-available dressings. In addition, it will save the caregiver's time to apply the vacuum therapy dressing to this area. Without assembly the separated parts of the dressing as the current commercially-available dressings, the embodiment of our invention may be dressed within 3 minutes by 3 easy steps, i.e. putting the dressing on the wound, having the pendent membrane adhere to the healthy skin and the opening, and connecting the tube to the negative pressure source.

As mentioned in the “What is claimed” part, our embodiments covers all the body regions except for the genital, the head, the hand and the face. One of the reasons we don't include embodiments to cover those said regions is because the circulation of them are very good and subsequently nearly no chronic wounds or infectious wounds are formed in these said regions under the treatment of conventional gauze changing. As is known to all, the vacuum therapy benefits chronic wounds and infectious wounds. Besides, it is not practical to cover eyes and mouth, and it is potentially to impair the circulation of the fingers when the whole hand is covered by the vacuum dressings, e.g. V.A.C.® GranuFoam™ Hand Dressing (KCI Medical Ltd), application of which squeeze the fingers together.

Features of the invention will become apparent to those skilled in the art upon consideration of the following detailed description of preferred embodiments exemplifying the best mode of carrying out the invention as presently perceived.

### BRIEF DESCRIPTION OF THE SEVERAL VIEWS OF THE DRAWING

FIG. 1 and FIG. 2 are cross-sectional views of a portion of the vacuum wound dressing according to the present invention;

FIG. 3 is a top view of the beehive-like isolation membranes of the body contour-fit layer;

FIG. 4, FIG. 5 and FIG. 6 are the embodiments of the suction tube layout that may be used in the vacuum wound dressing;

FIG. 7 is a perspective view of one embodiment of the vacuum wound dressing showing the dressing covers the foot, the ankle, the leg, the knee and part of the thigh;

FIG. 8 is a perspective view of one embodiment of the vacuum wound dressing showing the dressing covers the foot and the ankle;

FIG. 9 is a perspective view of one embodiment of the vacuum wound dressing showing the dressing covers the leg and the knee;

FIG. 10 is a perspective view of one embodiment of the vacuum wound dressing showing the dressing covers the knee and part of the thigh;

FIG. 11 is a perspective view of one embodiment of the vacuum wound dressing showing the dressing covers the knee;

FIG. 12 is the cross-sectional view taken along the line 12-12 of FIG. 11;

FIG. 13 is a perspective view of one embodiment of the vacuum wound dressing showing the dressing covers the forearm, the elbow and part of the arm;

FIG. 14 is a perspective view of one embodiment of the vacuum wound dressing showing the dressing covers the forearm, and part of the elbow;

FIG. 15 is a perspective view of one embodiment of the vacuum wound dressing showing the dressing covers the elbow;

FIG. 16A and FIG. 16B are the front and the back perspective view of one embodiment of the vacuum wound dressing showing the dressing covers both of the hips, the perineal region, part of both thighs and part of the abdomen;

FIG. 17A and FIG. 17B are the front and the back perspective view of one embodiment of the vacuum wound dressing showing the dressing covers one side of hips and part of the other, the perineal region, part of both thighs and part of the abdomen;

FIG. 18 is the perspective view of the genital protective structure which is used in the embodiments in FIG. 16A, FIG. 16B, FIG. 17A and FIG. 17B;

FIG. 19 is the exploded view of the urinary catheter hole and the rubber stopper;

FIG. 20A and FIG. 20B are the front view and the back perspective view of one embodiment showing the dressing covers the shoulder, the armpit, part of the arm, part of the chest and part of the back;

FIG. 21A and FIG. 21B are the front view and the back perspective view of one embodiment showing the dressing covers the shoulder, the armpit, part of the arm, the back, the chest, part of the abdomen and part of the hips;

FIG. 22 is the perspective view of one embodiment showing the dressing covers part of the arm;

FIG. 23 is the perspective view of one embodiment showing the dressing covers part of the forearm;

FIG. 24 is the perspective view of one embodiment showing the dressing covers part of the thigh;

FIG. 25 is the perspective view of one embodiment showing the dressing covers part of the leg;

FIG. 26 is the perspective view of one embodiment showing the dressing covers part of the chest and part of the abdomen;

FIG. 27 is the perspective view of the embodiment showing the dressing covers part of the back;

FIG. 28A and FIG. 28B are the front and the side perspective views of one embodiment showing that the dressing covers the flank area of the trunk.

### DETAILED DESCRIPTION OF THE INVENTION

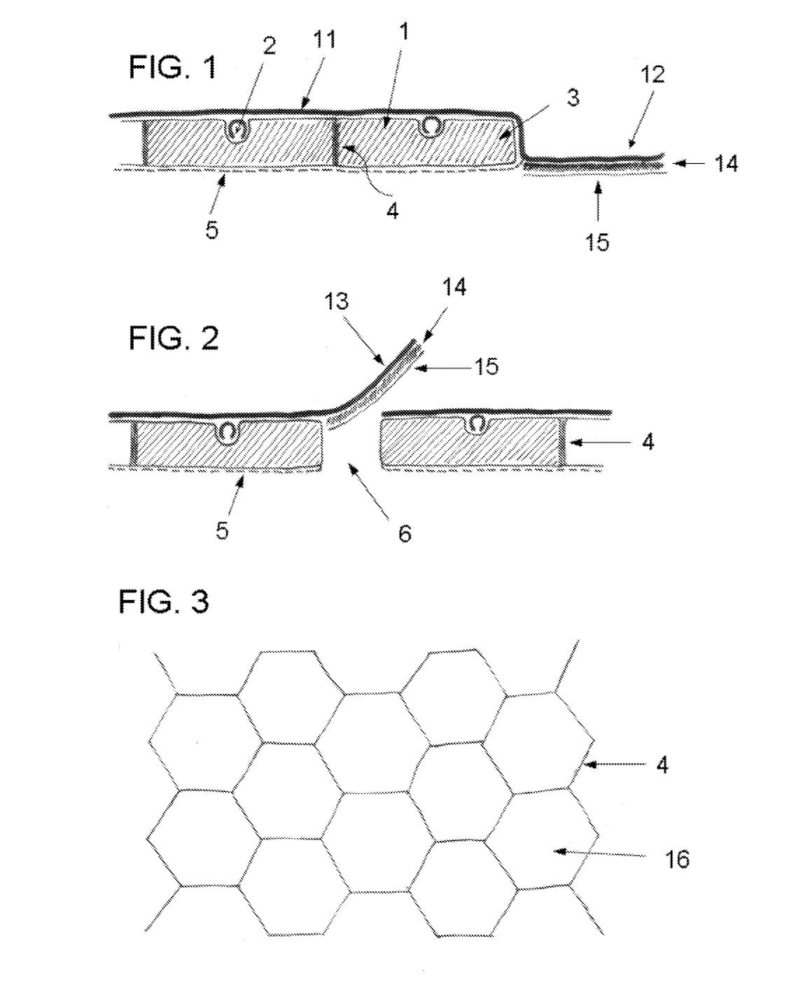

The cross-sectional views of the part of the vacuum wound dressing 1 are shown in FIG. 1 and FIG. 2. Particularly, FIG. 1 shows the marginal part of the said dressing and FIG. 2 shows the part of the dressing 1 by the sides of the opening 6. As shown in FIG. 1 and FIG. 2, the vacuum wound dressing 1 comprises an outer layer 11, a body contour-fit layer 3, a wound contacting layer 5, a tubing system 2, the pendent membranes 12&13 with viscous adhesive 14 and the protective membranes 15. All these parts are integrated into one piece.

The said outer layer 11 is thin and flexible, and it is airproof and waterproof. It maybe water vapor permeable. It is made of the medical grade synthetic materials such as nylon, rayon and polyester. It is within the scope of this disclosure, however, to include an outer layer made of any type of thin, flexible, airproof and waterproof material. The pendent membranes 12&13 are the continuation of the outer layer 11. A layer of adhesive 14 is distributed on one side of the pendent membranes. The said adhesive 14 is covered by the protective membranes 15, which can be easily peeled off without disturb the adhesive 14. However, it is within this disclosure to include a pendent membrane which is made of any materials suitable for this application.

The body contour-fit layer 3 is molded or tailored to fit the body region(s) which it is designed to cover. The said body contour-fit layer 3 is made of multi-pore materials, such as the polyurethane (PA) foam, the polyvinyl alcohol (PVA) foam, gauze, or any other materials. illustratively, the thickness of the said body contour-fit layer 3 is 1.6 cm. It is within the scope of this disclosure, however, to include the body contour-fit layer 3 having the various thicknesses suitable for the present application. The said body contour-fit layer is divided by the airproof and waterproof membrane 4, which is called the isolation membrane 4 thereafter in this application, into the obsolete cells 16. The said obsolete cells 16 of the body contour-fit layer 3 are shown in FIG. 3, where they are in regular hexagon shape and are separated by the said isolation membranes 4. Illustratively, the longest diagonal of the obsolete cells 16 is 8 cm and the thickness of the isolation, membrane 4 is 1 mm. However, it is within the scope of this disclosure for the obsolete cells to be any suitable shape and size. And it is within the scope of this disclosure for the isolation membrane 4 to have various suitable thicknesses suitable for the present application.

The wound contacting layer 5 is under the body contour-fit layer 3. The said wound contacting layer 5 is made of thin, flexible and non adherent gauze with multi pores. The non adherent gauze doesn't adhere to the tissue, thus decrease the pain during the dress changing. Many companies manufacture the non adherent gauze, such as Kendall Telfa non adherent dressing. The said wound contacting layer 5 may be other non adherent dressings made of synthetic materials, such as 3M™ Tegaderm™ or Mapitel (Mölnlycke Health Care US, LLC). It is within the scope of this closure, however, to include the wound contacting layer 5 made of any type of thin, flexible, non adherent and multi-pore materials suitable to the present application. Illustratively, the thickness of the said wound contacting layer 5 is 0.3 mm, the diameter of the said pores is 1 mm and the density of the pores is 60-90/cm². However, it is within the scope of this disclosure to include the wound contacting layer 5 having any suitable thickness suitable to the present application, and it is within the scope of this disclosure to include the wound contacting layer 5 with any suitable size and density of the pores. And it is also within the scope of this disclosure for the wound dressing to have a non-adherent body contour-fit layer 3 and no contacting layer 5.

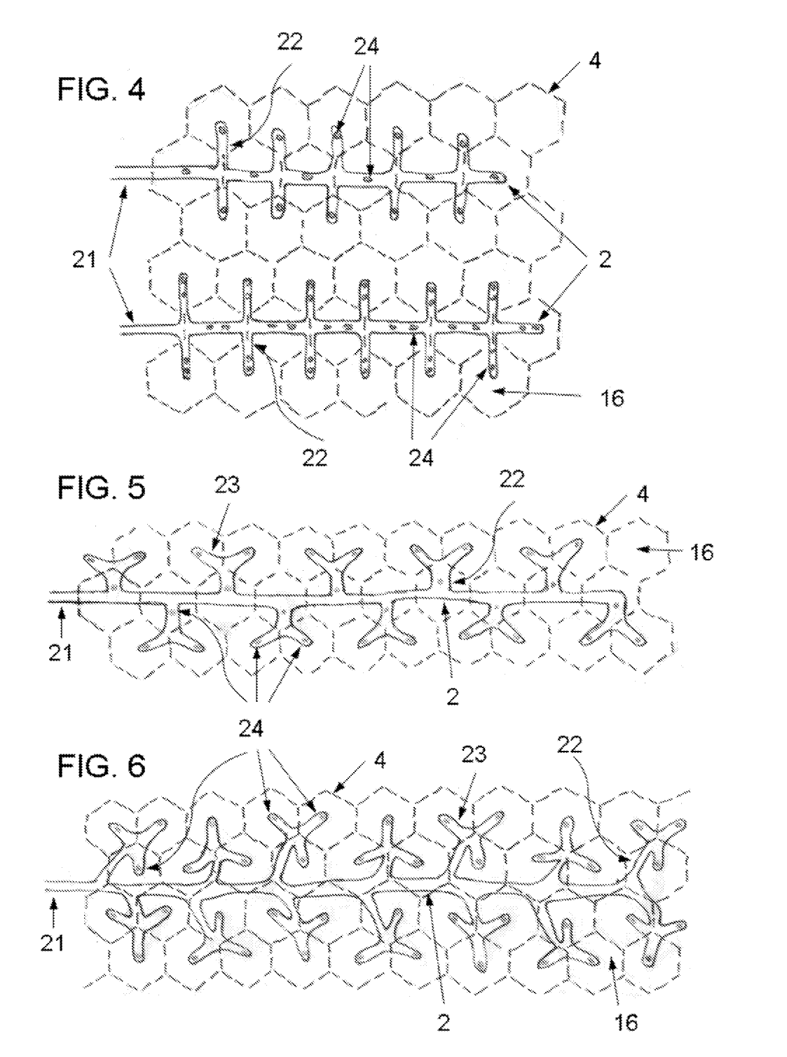

As shown in FIG. 1 and FIG. 2, the tubing system 2 locates under the outer layer 11, and is partly buried in the body contour-fit layer 3. The different types of layouts of the tubing system are shown in FIG. 4, FIG. 5 and FIG. 6, and all the said layouts are branch-like. It is within the scope of this disclosure, however, to have the tubing system locating on the said body contour-fit layer 3 or totally buried in the said body contour-fit layer 3. The main tube 21 or several main tubes 21 joined together connect with the negative pressure source. The thick branch tubes 22 connect with the main tubes 21 to deliver the negative pressure to thin branch tubes 23 (FIG. 5 and FIG. 6) or to the isolate cells 16 (FIG. 4 and FIG. 5) through holes 24. The thin branch tubes 23 connect with the thick branch tubes 22 to deliver the negative pressure to the isolate cells 16 (FIG. 5 and FIG. 6) through holes 24. Illustratively, the internal diameter of the said main tubes 21 is 7 mm, that of the said thick branch tubes 22 is 6.5 mm, that of the thin branch 23 tubes is 6 mm and that of holes 24 is 5 mm. However, it is within the scope of this disclosure to include the diameter of the main tubes 21, the thick branch tubes 22, the thin branch tubes 24 and the holes 24 having any sizes suitable to current application. Illustratively, the thickness of the tube wall is 0.7 mm for the main tubes 21, 0.6 mm for the thick branch tubes 22 and 0.5 mm for the thin branch tubes 23. It is within the scope of this application, however, to include the tube walls of main tubes 21, the thick branch tubes 22 and the thin branch tubes 23 having various thicknesses suitable for the present application. It is also within the scope of this application to include branch-like tubing systems 2 having any kinds of layouts suitable to the current application. And it is within the scope of this application to include more than one hole 24 for each obsolete cell 16.

Further, it is within the scope of this disclosure to include several main tubes 21 in one vacuum wound dressing 1. The embodiment shown in FIG. 4 has two main tubes 21, and these two said main tubes join together outside of the dressing 1 and then connect to the negative source. However, it is within this disclosure to include several main tubes 21 joining together in the wound dressing 1.

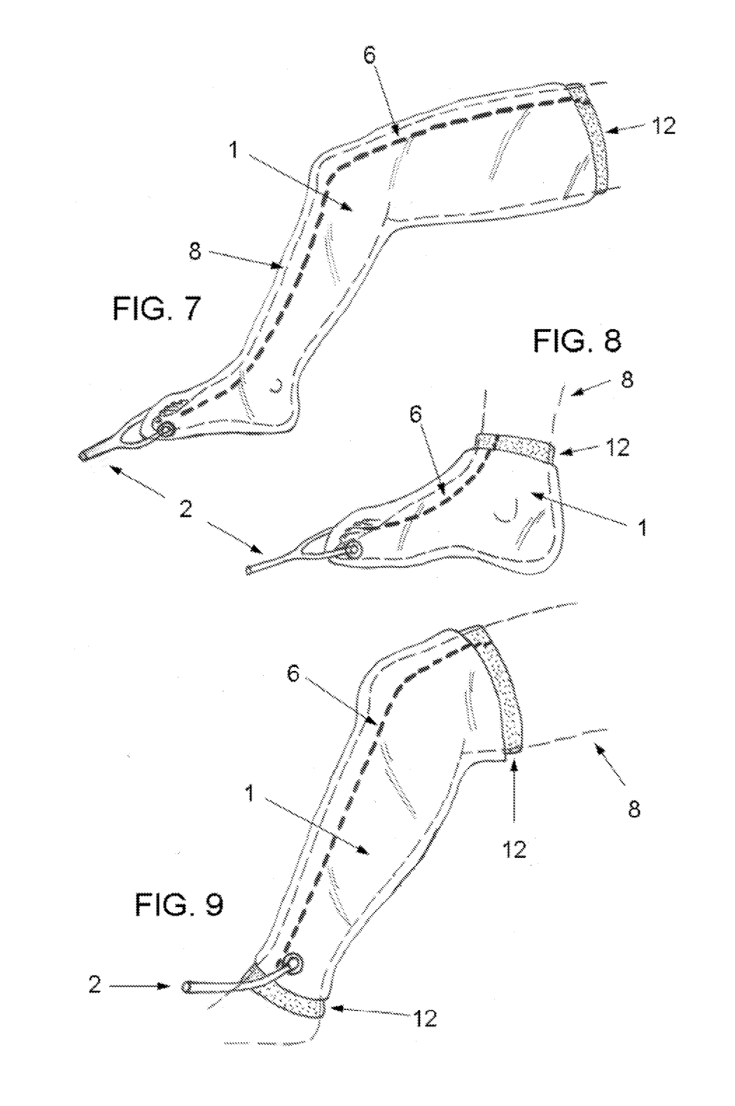

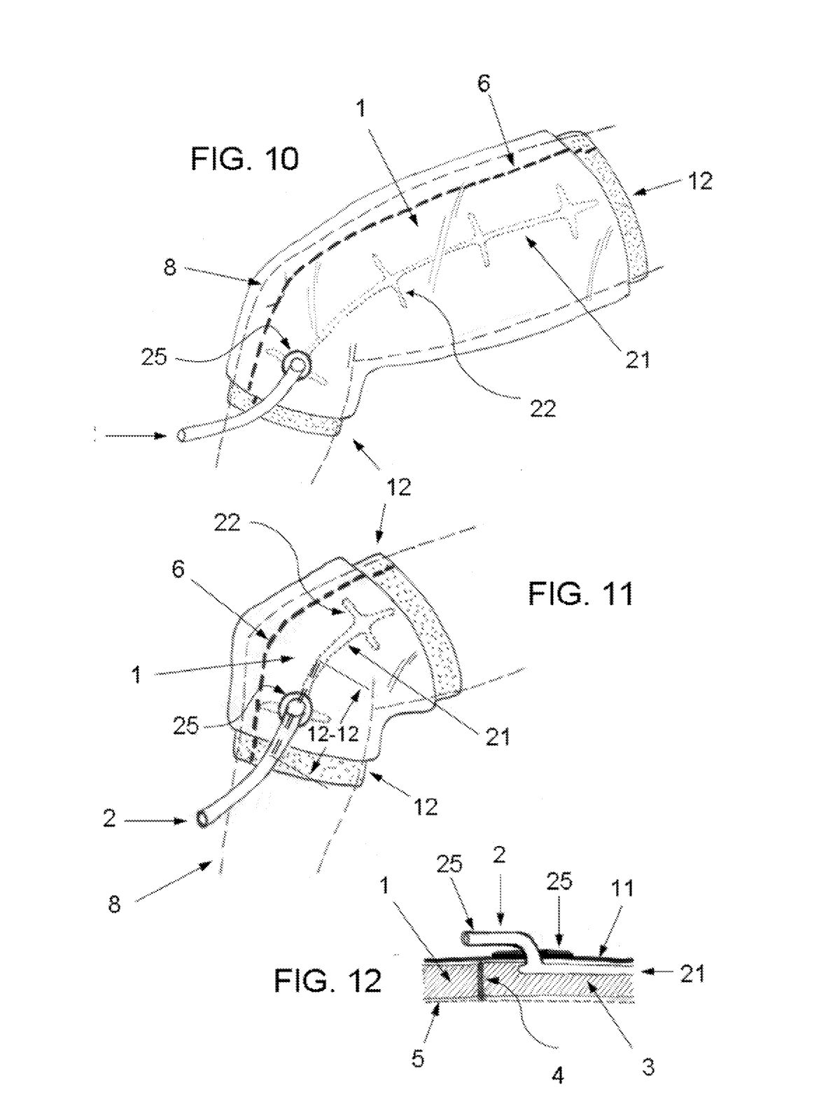

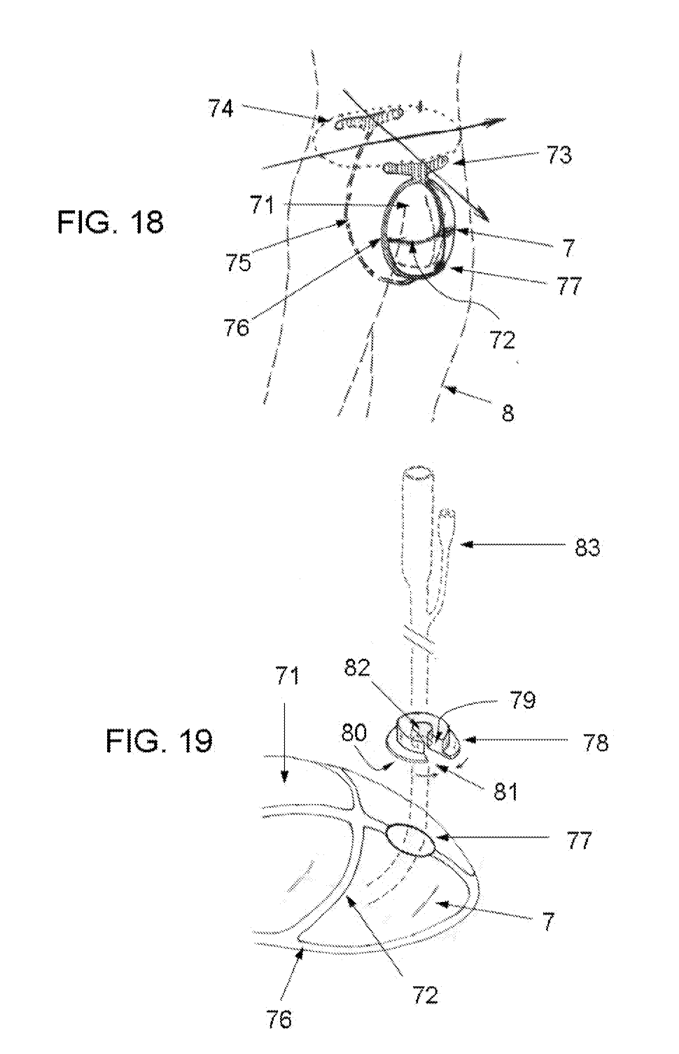

Embodiments shown in the figures FIG. 7 to FIG. 21B (except for the FIG. 12, FIG. 18 and FIG. 19) are the perspective view of the vacuum wound dressings 1 covering different body region(s). All the said embodiments surround the body region(s) they cover, and they have the opening(s) 6 via which they are put on the said body region(s). All the said embodiments have the pendent membranes 13 with adhesives on one side of the opening 6 which are used to seal the opening 6, however the said pendent membranes are not shown in the said figures (form FIG. 7 to FIG. 21B except for the FIG. 12, FIG. 18 and FIG. 19). All the said embodiments have the tubing systems 2, however the said tubing system 2 are not shown in the said figures except FIG. 10 and FIG. 11. All the embodiments described in this application cover one or more than one human regions. However, it is within this disclosure to include the body contour-fit vacuum wound dressings 1 covering part of the human body region in a surrounded way. Further, it is within this disclosure to include the vacuum wound dressing 1 covering any combination of adjacent human body regions.

Human body regions mentioned hereafter in this application may include one or several the known anatomical body regions of human. The body regions mentioned in this application include the neck (the cervical region), the shoulder (the acromial region), the chest (including the thoracic regions and the sternal region), the abdomen (including the abdominal region, the umbilical region, the pelvic region and the inguinal region), the back (including the vertebral region, the scapular region and the lumbar region), the hips (including both the gluteal regions and the sacral region), the perineal region (region between the anus and the external genitalia), the thigh (the femoral region), the knee (the patellar region and the popliteal region), the leg (including the crural region and the sural region), the ankle (the tarsal region), the foot (including the plantar region, the calcaneal region, the digitals, and the hallux, the armpit (the axillary region), the arm (including the antecubital region and the cubital region), the elbow (including the antecubital region and the cubital region), the forearm (including the antebrachial region and the brachial region), and the wrist (the carpal region).

The body contour-fit layer 3 and the suction tubing system 2 in all said embodiments are molded or tailored to fit the contour of the body region(s) which they cover. The dressings 1 covering different body region(s) are either mass-manufactured into different sizes or custom-manufactured. When the wound change the body contour significantly, the wound dressing will be custom-manufactured.

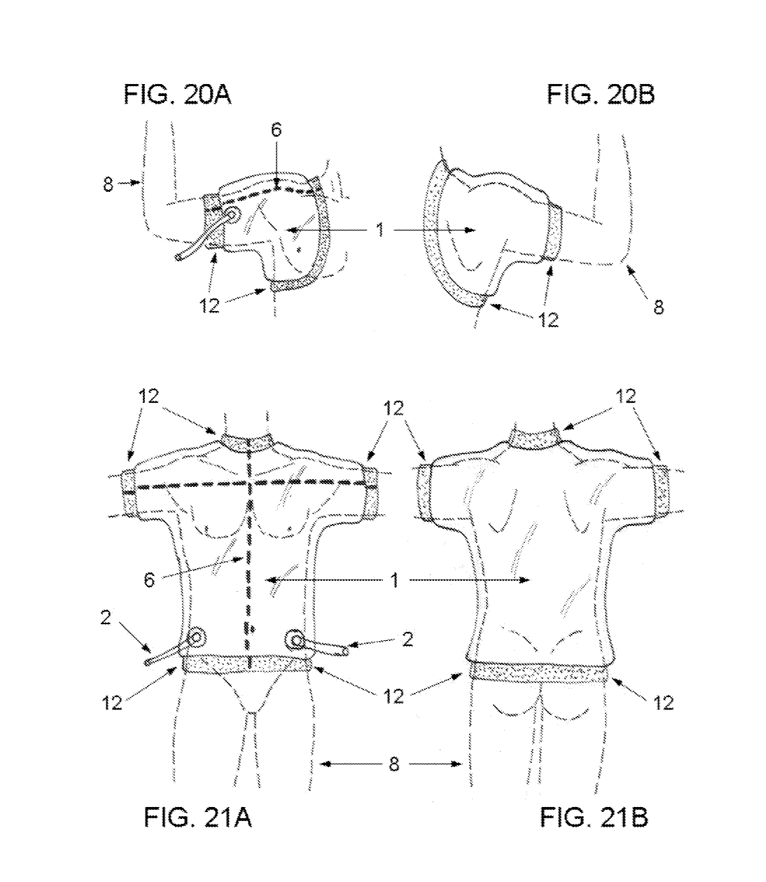

The embodiments shown in some of the figures of this application fit the covered joints at certain position. For example, the embodiment shown in FIG. 10 fit the knee with 40 degree of flexion, and that shown in FIG. 20A fit the shoulder with 90 degrees deduction. However, it is within the scope of this disclosure to include the vacuum wound dressings 1 to fit the covered joints at any suitable positions.

The openings 6 for some of the embodiments described in this application are at the front or the lateral of the vacuum wound dressing 1, and they are in the shapes of curved or straight lines. However, it is within the scope of this disclosure to include the opening(s) 6 of any shape and at any suitable position in the vacuum wound dressing 1.

Embodiments shown in FIGS. 7, 8, 9, 10 and 11 are the vacuum wound dressings 1 to cover the most of the lower limb (FIG. 7), the foot and the ankle (FIG. 8), the knee and the leg (FIG. 9), most of the thigh and the knee (FIG. 10), and the knee (FIG. 11). The dressings 1 surrounded the body region.

As shown in FIG. 12, the tubing system 2 includes a suction tube 25 which connect the main tube(s) 21 to the negative source. At the position where the suction tube 25 enters the outer membrane 11, there is a round rubber support disk 25. The said support disk 25 is fixed on the outer layer 11 and on the suction tube 25, thus it will disperse the mechanical forces when the suction tube 25 is accidently pulled. Illustratively, the said round rubber support disk is 2 mm in thickness and 6 cm in diameter. However, it is within this disclosure to include, a support disk 25 having any shapes and any dimension suitable for the current application. And it is also within this disclosure to have a support disk 25 made of any material suitable for current application.

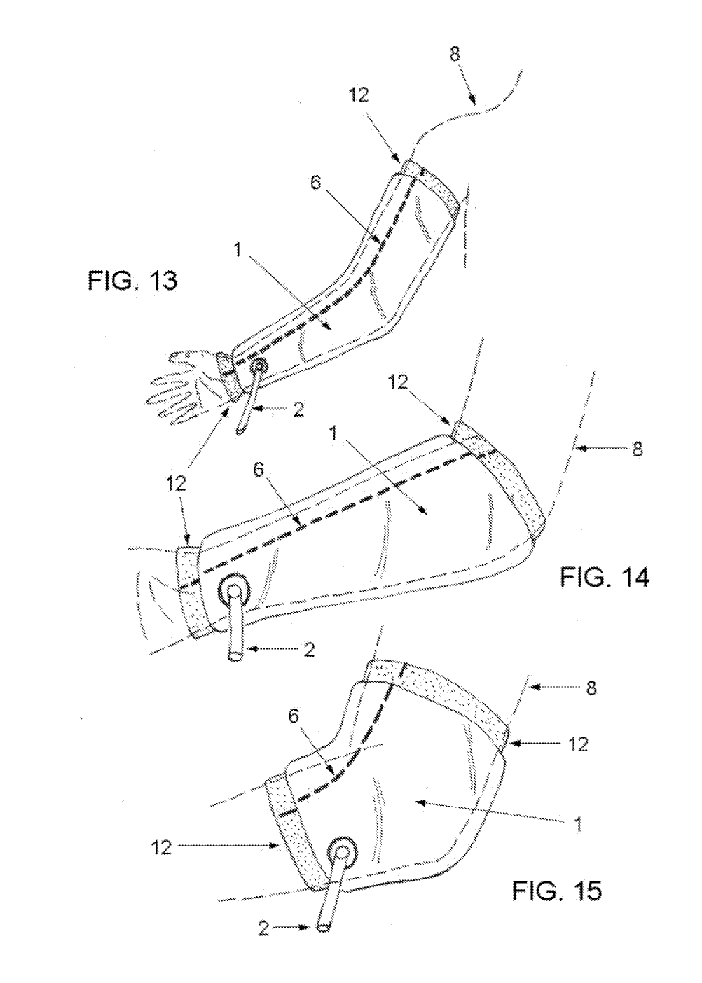

The embodiments shown in FIGS. 13, 14 and 15 are the vacuum wound dressings 1 covering the whole upper limb, the forearm, and the elbow respectively. The said wound dressings 1 surround the body region which they cover.

Please note that only one perspective view of each embodiment is shown in the FIGS. 7 to 15 (except for FIG. 12). On the unshown opposite side of the each embodiment in the said FIGs, the wound dressings end at the same level as shown in the corresponding FIGs.

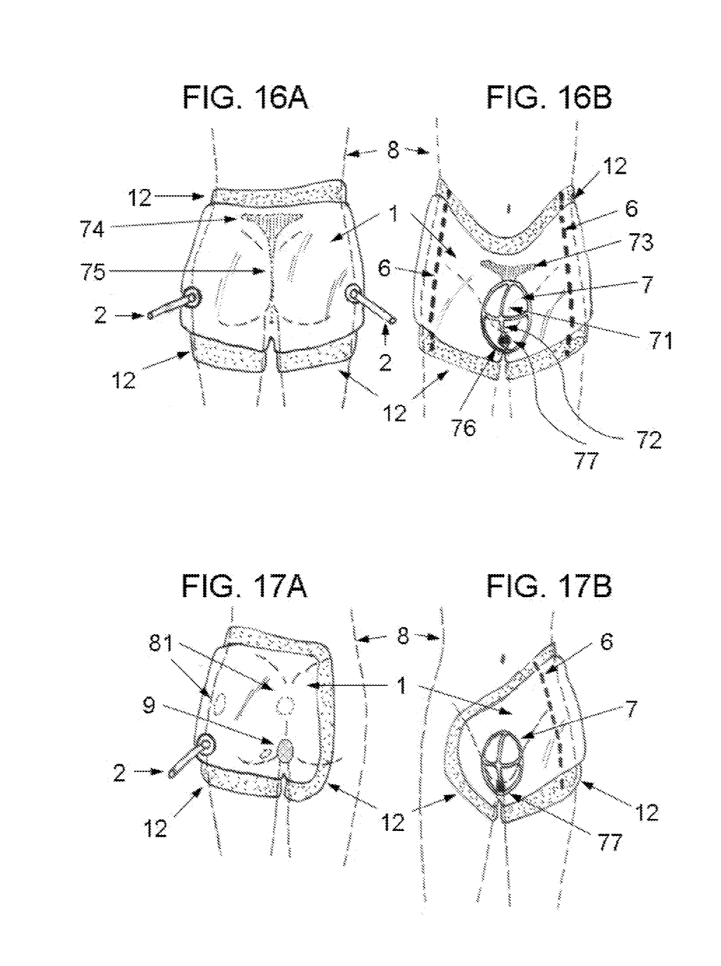

The embodiments shown in FIG. 16A and FIG. 16B are the front and the back perspective view of the vacuum wound dressing 1 covering both the hips, part of the both thighs, the perineal region and part of the abdomen. There are two openings 6 for the dressing 1. The vacuum wound dressing 1 may only cover one side and part of the other side of the hip as shown in FIG. 17A (the back view) and FIG. 17B (the front view).

As shown in FIG. 17A, there is a color-changing stool indicator 9 on the vacuum wound dressing 1 over the anal. The outer membrane 11 over the said stool indicator 9 is transparent and no body contour-fit layer 3 over the said stool indicator 9. The said color-changing stool indicator 9 is a thin and flexible membrane made of any materials which will change color when encounter the stool, but will not change color when encounter with wound fluid.

The embodiments shown in FIG. 16A, FIG. 16B, FIG. 17 A and FIG, 17B include a genital protective structure 7 which comprises a transparent dome 71, the front anchor 73, the curved strut 75 and the back anchor 74. The said genital protective structure 7 is integrated with the vacuum wound dressing 1 into one piece. Only the transparent dome 71 is shown in FIG. 17 B, and the other parts of the genital protective structure 7 are not shown, only the genital protective structure 7 is shown in FIG. 18 and the other parts of the dressing 1 is not shown.

The transparent dome 71, the curved strut 75, the front anchor 73 and the back anchor 74 is made of transparent plastic materials, and don't collapse upon the negative pressure applied to the vacuum wound dressing 1. It is within the scope of this disclosure to include the said parts of the genital protective device 7 made of any suitable materials.

The said genital protective structure 7 includes a cross-shaped curved strut 72 and an elliptic base 76, both of which are made of transparent rigid plastic or synthetic material, to support the transparent dome 71. However, it is within this disclosure, to include a transparent and rigid dome 71 which is strong enough to support itself and thus no cured strut 72 and elliptic base 76 are needed. Illustratively, the transparent dome is 18 cm in height, 8 cm in width and 1.5 mm in thickness. It is in the scope of this disclosure, however, to include the dome have different dimensions to suit the size of the genitals of different people or different genders. It is also in the scope of this disclosure to include different type of support or no support in the transparent dome 71. Further, it is in the scope of this disclosure to include a transparent rigid structure with any kind of shapes to replace the said transparent dome 71.

The front anchor 73 and the back anchor 74 have adhesives and are under the body contour-fit layer 3. The wound contacting layer 5 ends on the margins of the said anchors 73&74. The said adhesive is protected by protective membranes. Illustratively, the front anchor 73 and the back anchor 74 are adhered to the skin over the pubis and the sacrum respectively to secure the transparent dome 71 over the genital of the patient it is within this disclosure, however, to include the front anchor 73 and the back anchor 74 adhering to the skin in any suitable regions the patient. Further, it is within this disclosure to include the front anchor 73 and the back anchor 74 locating over the body contour-fit layer 3 or the outer layer 11.

The curved strut 75 is under the outer layer 11 but above the body contour-fit layer 3. The outer layer 3 ends on the base 76 of the transparent dome 71. The body contour-fit layer 3 ends along the base 76 of the said transparent dome 71. The genital is only covered by the said transparent dome 71 and is easily to be inspected. It is within this disclosure, however, to include the curved strut 75 and the base 76 locating at any positions relative to the parts of the vacuum wound dressing 1.

There is a hole 77 for the urinary catheter 83 on the transparent dome 7 as shown in FIG. 16B, FIG. 17B, FIG. 18 and FIG. 19, through which the commercially available urinary catheters 83 can pass. Illustratively, the hole 77 for the urinary catheter 83 is round and 2 cm at the diameter. It is within the scope of this disclosure to include the hole 77 for the urinary catheter 83 having any type of shape and variable size suitable for current application.

As shown in FIG. 19, an elastic rubber stopper 78 with opening 81 is used to secure the catheter and seal the hole 77 for the urinary catheter 83. The said rubber stopper 78 has a hole 82 in the center, with the diameter 1 mm smaller than that of the outer diameter of the urinary catheter 83. The shape of the elastic rubber stopper 78 is shown in FIG. 19. In the embodiment shown in FIG. 19, the diameter of the upper part of the said rubber stopper 78 is 22 mm, which is 2 mm larger than that of the urinary catheter hole 77 on the transparent dome 71, the diameter of the lower part of the said rubber stopper 77 is 30 mm which is 10 mm larger than that of the urinary catheter hole 77. However, it is within this disclosure to include a stopper with any shape and any size, and made of any material suitable to this application.

The facades 79 on both sides of the opening 81, the wall of the hole 82 and the bottom 80 of the elastic rubber stopper 78 have adhesives, which is protected by the protective membranes (not shown in FIG. 19). Upon application of the wound dressing 1, the dressing is put on the patient through the opening 6 with the transparent dome on the right position and the stool indicator exactly on the anal. The front anchor 73 and the back anchor 74 are adhered to the skin of the patient to secure the position of the genital protective device 7. Then pass the urinary catheter 83 through the urinary catheter hole 77. Put the rubber stopper 78 around the urinary catheter 83 through the opening 81, peel off the all the protective membranes on the rubber stoppers 78, and squeeze the rubber stopper 78 to adhere it to the urinary catheter 83 and to close the opening 81. Then press the rubber stopper 78 on the hole 77 for the urinary catheter 83. Then the said hole for the urinary catheter 77 is sealed.

Embodiment shown in FIG. 20A and FIG. 20B is the front and the back perspective view of the vacuum wound dressing 1 covering the shoulder, the armpit, part of the arm, part of the back and part of the chest.

Embodiment shown in FIG. 21A and FIG. 21B is the front and the back perspective view of the vacuum wound dressing 1 covering part of the neck, the chest, the back, the shoulder, the armpit, part of the arms, part of the abdomen and part of the hips. The opening of this said dressing 1 is illustrated in FIG. 21A. However, it is within the scope of this disclosure for the said wound dressing 1 to have a suitable number of openings and to have the opening located at any suitable positions.

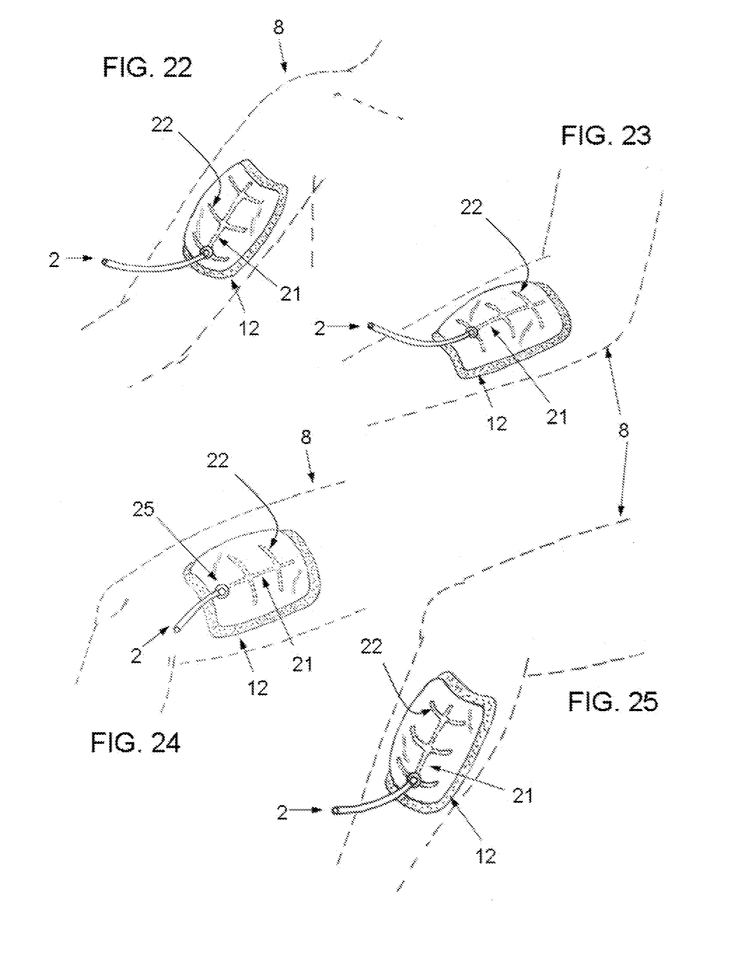

Unlike the embodiments shown in FIG. 7 to FIG. 21 which surround and cover the corresponding body region(s), the embodiments shown in FIG. 22 to FIG. 28 don't surround the body region(s) they cover. Therefore there are no openings 6 for these embodiments. In these said embodiments, both the body contour-fit layer 3 and the suction tubing system 2 are molded or tailored to fit the body contour which they cover. The said embodiments are either mass-manufactured into different sizes or custom-manufactured based on the patient's be region contours, and the contour of wounds.

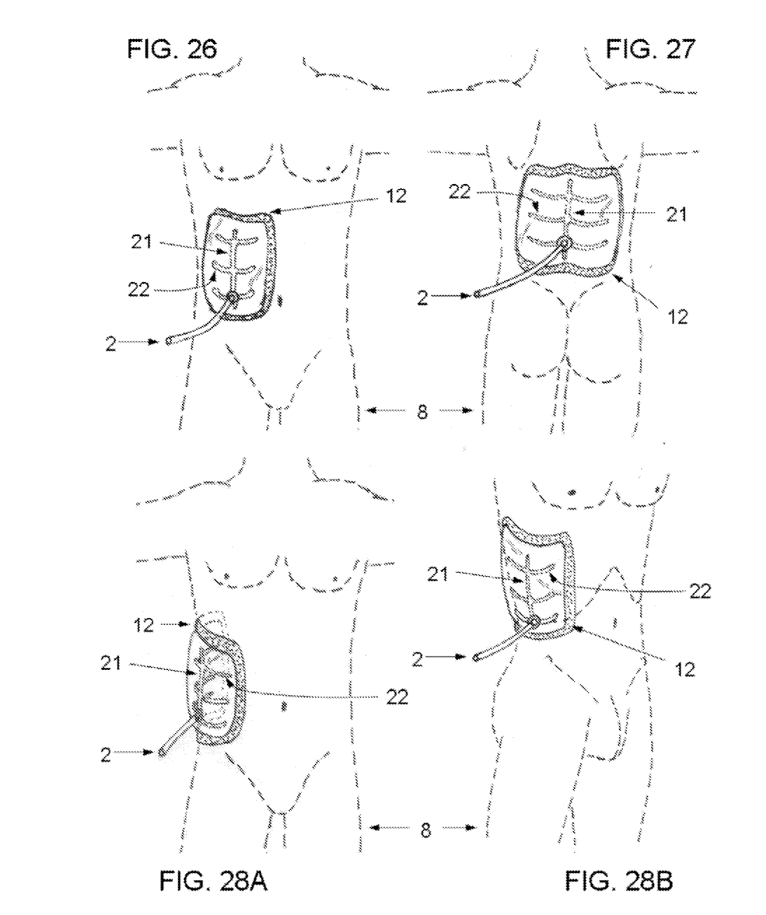

Illustratively, the dressing 1 shown in FIG. 22 is 8 cm in width and 16 cm in length, that shown in FIG. 23 is 4 cm in width and 8 cm in length, that shown in FIG. 24 is 15 cm in width and 25 cm in length, that shown in FIG. 25 is 10 cm in width and 15 cm in length, that shown in FIG. 26 is 15 cm in width and 25 cm in length, that in FIG. 27 is 25 cm in width and 30 cm in length, and that in FIG. 28A and FIG. 28B is 20 cm in width and 25 cm in length. These dressings fit the body contour of arm, forearm, thigh, leg, chest or abdomen, back and the flank. However, it is within the scope of this disclosure to include the wound dressing 1 fitting all the human body regions except head, face, hand and genital. Further, it is within the scope of this disclosure to include the wound dressing 1 having any size and any shape suitable to the current application.

Although the curvature of human body changes from point to point, and body contour of different body regions are different, some parts of different human body regions share similar curvature. For example, the front part of the thigh, the flank side of the chest and back, and the flank side of the waist share similar curvature. Therefore, it is within this disclosure for the wound dressing having a certain curvature to fit parts of different body regions with similar curvature.

Although this invention has been described in detail with reference to certain embodiments, variations and modifications exist within the scope and spirit of the invention as described and defined in the following claims.

What is claimed is:

1. A wound dressing for vacuum therapy comprising:
a body contour-fit layer which is made of multi-pore material and is casted or tailored to fit and cover part of one, one or several human anatomical regions; a thin flexible outer layer which is airproof and waterproof; a wound contacting layer which is thin non-adherent to the wound, and has multi pores; a branch-like suction tubing system which connect the body contour-fit layer to the vacuum source, and is also molded to conform to the body contour; and the pendent membranes with viscous adhesive on all the margins of the dressing.

2. A wound dressing according to claim 1 wherein all the parts of the wound dressing are integrated to one piece.

3. A wound dressing according to claim 1 wherein the body contour-fit layer is separated by the waterproof and airproof membranes into obsolete cells.

4. A wound dressing according to claim 1 wherein the body contour-fit layer and the tubing system are molded or tailored to fit and cover part of or one of the body region listed below: the neck, the shoulder, the chest, the abdomen, the back, the hips, the perineal region, the thigh, the knee, the leg, the ankle, the foot, the armpit, the arm, the elbow, the forearm and the wrist.

5. A wound dressing according to claim 1 wherein the body contour-fit layer is casted or tailored to fit and cover 2 or several adjacent body regions mentioned in claim 4, with or without one or several of the said regions to be partial.

6. A wound dressing according to claim 1, which surrounds part of the limbs or the trunk, comprises one or several openings.

7. A wound dressing according to the claim 1, which needs to include the genital under the dressing, comprises a genital protective structure.

8. The genital protective structure according to the claim 7 includes a hole for the urinary catheter.

9. The wound dressing according to the claim 7 comprises an elastic rubber stopper to secure the urinary catheter and to seal the hole on the genital protective structure.

10. The wound dressing according to the claim 7 comprises a color-changing stool indicator.

11. The wound dressing for the certain body region(s) according to the claim 4 and claim 5 has different sizes to fit the said region(s) with different dimensions.

12. The wound dressing for the certain body region(s) according to the claim 4 and claim 5 can also be custom-manufactured according to the dimension of the patient's said body region(s).
